(19) United States
(12) Patent Application Publication
(10) Pub. No.: US 2024/0295567 A1
(43) Pub. Date: Sep. 5, 2024

(54) HYPOXIA BIOMARKER AND USE THEREOF

(71) Applicant: Kyoto University, Kyoto-shi, Kyoto (JP)

(72) Inventors: Hiroshi HARADA, Kyoto-shi, Kyoto (JP); Minoru KOBAYASHI, Kyoto-shi, Kyoto (JP); Tatsuya SUWA, Kyoto-shi, Kyoto (JP)

(21) Appl. No.: 18/574,504

(22) PCT Filed: Jun. 20, 2022
(86) PCT No.: PCT/JP2022/024612

§ 371 Date: Dec. 27, 2023

(30) Foreign Application Priority Data
Jun. 29, 2021 (JP) 2021-108196

(51) Int. Cl. G01N 33/68; G01N 33/50

(52) U.S. Cl. CPC G01N 33/6893; G01N 33/5008; G01N 2333/811; G01N 2800/56; G01N 2800/70

(57) ABSTRACT

Provided are: a novel marker useful for measuring hypoxia or predicting the risk of exacerbation of a hypoxia-related pathological condition; a hypoxia measurement method or a method for predicting the risk of exacerbation of a pathological condition using the marker as an indicator; a composition for measuring hypoxia or predicting the risk of exacerbation of a pathological condition; a method for screening a candidate substance for a therapeutic agent for a hypoxia-related pathological condition; a composition for ameliorating a hypoxia-related pathological condition; and the like. A method for measuring hypoxia in a subject or a method for predicting the risk of exacerbation of a pathological condition, each method comprising the step of measuring the amount of SPINK1 in a sample collected from a subject. Use of SPINK1 as a biomarker for measuring hypoxia or for predicting the risk of exacerbation of a pathological condition. A composition for measuring hypoxia or predicting the risk of exacerbation of a pathological condition, the composition comprising a SPINK1 detection reagent. A method for screening a candidate substance for a therapeutic agent for a hypoxia-related pathological condition, the screening being performed by using, as an indicator, whether or not the candidate substance can inhibit SPINK1. A composition for ameliorating a hypoxia-related pathological condition, the composition comprising a SPINK1 inhibitor.

### TECHNICAL FIELD

The present invention relates to a hypoxia biomarker and use thereof.

### BACKGROUND ART

A hypoxic state in vivo was heretofore known to be associated with various pathological conditions. For example, it is known that the inside of solid tumors has an area in a hypoxic state (an area at a low oxygen partial pressure) and that a hypoxic state is an important environmental factor that induces the resistance of cancer cells to radiotherapy and anticancer drug therapy. It is also said that the lower the oxygen concentration, the worse the pathological condition. Accordingly, in order to comprehend and predict pathological conditions and therapeutic efficacy on the pathological conditions, it is important to know, for example, the extent of hypoxic areas and the degree of hypoxia.

Today, hypoxic PET imaging using a probe, such as ¹⁸F-fluoromisonidazole, is known as a potential method for comprehending hypoxia in vivo. By using this method, the presence of a hypoxic area in vivo and the location and extent of the hypoxic area can be evaluated (Non-patent Literature (NPL) 1). However, this method has a problem in that it is difficult to use repeatedly. There is also another problem in that since the PET scanner itself is expensive, the number of facilities where this method can be performed is limited. Known as another method for comprehending hypoxia in vivo is, for example, an immunostaining method comprising sampling a part of tissue from a living body and targeting, for example, CA9 (carbonic anhydrase 9) or GLUT1 (glucose transporter 1) in the sampled tissue. However, since this method essentially requires the sampling of tissue fragments, the method has a problem in terms of always being highly invasive.

### CITATION LIST

### Non-Patent Literature

NPL 1: Koh, W. J. et al., Imaging of hypoxia in human tumors with [F-18] fluoromisonidazole, Int. J. Radiation Oncology Biol. Phys., 1992, vol. 22, pp. 199-212.

### SUMMARY OF INVENTION

### Technical Problem

An object of the present disclosure is to provide a novel marker useful for measuring hypoxia and predicting the risk of exacerbation of a hypoxia-related pathological condition; a method for measuring hypoxia using the marker as an indicator; a method for predicting the risk of exacerbation of a pathological condition using the marker as an indicator; a composition for measuring hypoxia or predicting the risk of exacerbation of a pathological condition; a method for screening for a therapeutic agent for a hypoxia-related pathological condition; a composition for ameliorating a hypoxia-related pathological condition; and the like.

### Solution to Problem

The present inventors conducted extensive research and found that the expression level and the secretion level of SPINK1 (serine protease inhibitor Kazal type I) increase in a hypoxia stimulus-dependent manner. The present inventors further found that SPINK1 is involved in the exacerbation of a pathological condition. The present inventors further found that SPINK1 is useful as a biomarker for hypoxia in a subject or as a biomarker for the exacerbation of a pathological condition. The present invention has been accomplished as a result of further research based on the above findings. The present invention includes, for example, the following embodiments.

Item 1. A method for measuring hypoxia in a subject, comprising measuring the amount of SPINK1 (serine protease inhibitor Kazal type I) in a sample collected from the subject.

Item 2. A method for predicting the risk of exacerbation of a pathological condition, comprising measuring the amount of SPINK1 in a sample collected from a subject.

Item 3. The method according to Item 1 or 2, wherein the sample is at least one member selected from the group consisting of blood, plasma, serum, urine, milk, saliva, cell specimens, and tissue specimens.

Item 4. Use of SPINK1 as a biomarker for measuring hypoxia or predicting the risk of exacerbation of a pathological condition.

Item 5: A composition for measuring hypoxia or predicting the risk of exacerbation of a pathological condition, the composition comprising a SPINK1 detection reagent.

Item 6. A method for screening a candidate substance for a therapeutic agent for a hypoxia-related pathological condition, the screening being performed by using, as an indicator, whether or not the candidate substance can inhibit SPINK1.

Item 7. A composition for ameliorating a hypoxia-related pathological condition, the composition comprising a SPINK1 inhibitor.

Item 8. The method according to Item 2 or 3, the use according to Item 4, the composition according to Item 5, the method according to Item 6, or the composition according to Item 7, wherein the pathological condition is at least one member selected from the group consisting of tumors and ischemic diseases.

### Advantageous Effects of Invention

The present invention can provide a method of measuring hypoxia and a method for predicting the risk of exacerbation of a pathological condition by using SPINK1 as an indicator. The present invention can provide a hypoxia biomarker useful for measuring hypoxia or predicting the risk of exacerbation of a pathological condition. Further, the present invention can provide a composition for measuring hypoxia or predicting the risk of exacerbation of a pathological condition, the composition comprising a SPINK1 detection reagent; a method for screening a candidate substance for a therapeutic agent for a hypoxia-related pathological condition, the screening being performed by using, as an indicator, whether or not the candidate substance can inhibit SPINK1; a composition for ameliorating a hypoxia-related pathological condition, the composition comprising a SPINK1 inhibitor; and the like.

### BRIEF DESCRIPTION OF DRAWINGS

FIG. 1 is a diagram showing examples of SPINK1 reported in the NCBI.

FIG. 2 is a diagram showing examples of SPINK1 reported in the NCBI.

FIG. 3 is a diagram showing examples of SPINK1 reported in the NCBI.

FIG. 4 is a diagram showing examples of SPINK1 reported in the NCBI.

FIG. 5 is a diagram showing examples of SPINK1 reported in the NCBI.

FIG. 6 is a diagram showing examples of SPINK1 reported in the NCBI.

FIG. 7 is a diagram showing examples of SPINK1 reported in the NCBI.

FIG. 8 is a diagram showing examples of SPINK1 reported in the NCBI.

FIG. 9 is a diagram showing examples of SPINK1 reported in the NCBI.

FIG. 10 is a diagram showing examples of SPINK1 reported in the NCBI.

FIG. 11 is a diagram showing examples of SPINK1 reported in the NCBI.

FIG. 12 is a diagram showing examples of SPINK1 reported in the NCBI.

FIG. 13 is a diagram showing examples of SPINK1 reported in the NCBI.

FIG. 14 is a diagram showing examples of SPINK1 reported in the NCBI.

FIG. 15 is a diagram showing examples of SPINK1 reported in the NCBI.

FIG. 16 is a diagram showing examples of SPINK1 reported in the NCBI.

FIG. 17 is a diagram showing examples of SPINK1 reported in the NCBI.

FIG. 18 is a diagram showing examples of SPINK1 reported in the NCBI.

FIG. 19 is a diagram showing examples of SPINK1 reported in the NCBI.

FIG. 20 is a diagram showing examples of SPINK1 reported in the NCBI.

FIG. 21 is a diagram showing examples of SPINK1 reported in the NCBI.

FIG. 22 is a diagram showing examples of SPINK1 reported in the NCBI.

FIG. 23 is a diagram showing examples of SPINK1 reported in the NCBI.

FIG. 24 is a diagram showing examples of SPINK1 reported in the NCBI.

FIG. 25 is a diagram showing examples of SPINK1 reported in the NCBI.

FIG. 26 is a diagram showing examples of SPINK1 reported in the NCBI.

FIG. 27 is a diagram showing examples of SPINK1 reported in the NCBI.

FIG. 28 is a diagram showing examples of SPINK1 reported in the NCBI.

FIG. 29 is a diagram showing examples of SPINK1 reported in the NCBI.

FIG. 30 is a diagram showing examples of SPINK1 reported in the NCBI.

FIG. 31 is a diagram showing examples of SPINK1 reported in the NCBI.

FIG. 32 is a diagram showing examples of SPINK1 reported in the NCBI.

FIG. 33 is a diagram showing examples of SPINK1 reported in the NCBI.

FIG. 34 is a diagram showing examples of SPINK1 reported in the NCBI.

FIG. 35 is a diagram showing one example of SPINK1 reported in the NCBI.

FIG. 36 is a diagram showing the results of SPINK1 mRNA expression in a hypoxia stimulus-dependent manner.

FIG. 37 is a diagram showing the results of SPINK1 mRNA expression and SPINK1 protein secretion in a hypoxia stimulus-dependent manner.

FIG. 38 is a diagram showing that SPINK1 reflects the amount of hypoxia in tumors.

FIG. 39 is a diagram showing that SPINK1 is secreted from cells in a hypoxic state and is present in or around the cells.

FIG. 40 is a diagram showing that an increase of SPINK1 contributes to the exacerbation of a pathological condition.

FIG. 41 is a diagram showing that SPINK1 can be used as an indicator of the exacerbation of a hypoxia-related pathological condition.

FIG. 42 is a diagram showing that an increase of SPINK1 contributes to exacerbation of a pathological condition.

FIG. 43 is a diagram showing that inhibition of SPINK1 can inhibit the exacerbation of a pathological condition.

FIG. 44 is a diagram showing that a SPINK1 high-value group has a lower overall survival rate than a SPINK1 low-value group.

### DESCRIPTION OF EMBODIMENTS

Embodiments included in the present disclosure are described below in more detail. In the present disclosure, the terms “comprise” and “contain” include the meanings of “substantially consisting of” and “consisting of.”

### Method for Measuring Hypoxia

The method for measuring hypoxia in a subject, which is included in the present disclosure, comprises the step of measuring the amount of SPINK1 (serine protease inhibitor Kazal type I) in a sample collected from a subject.

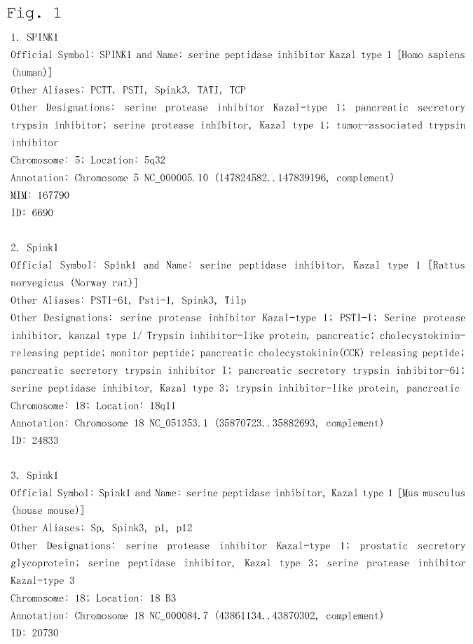

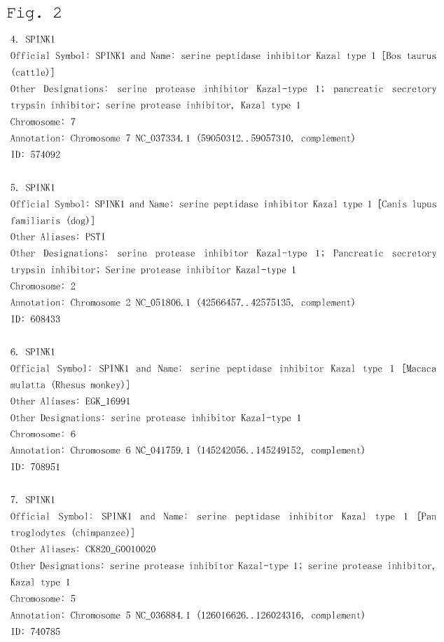

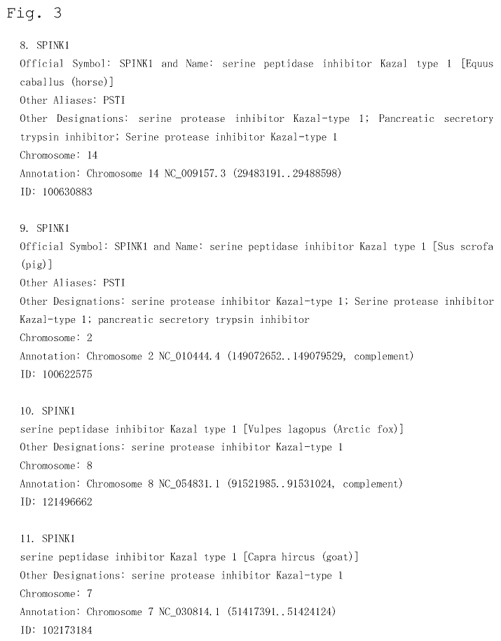

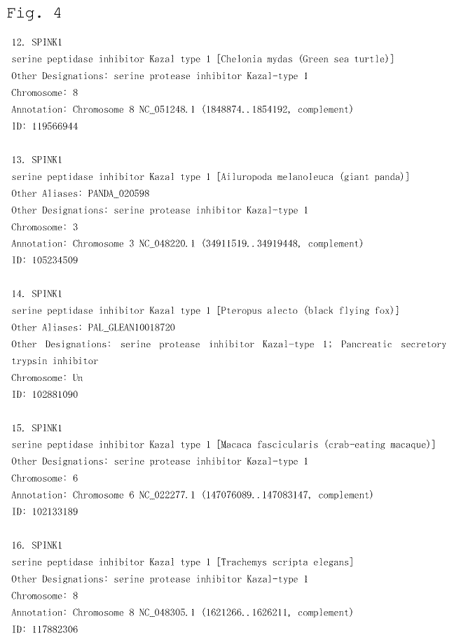

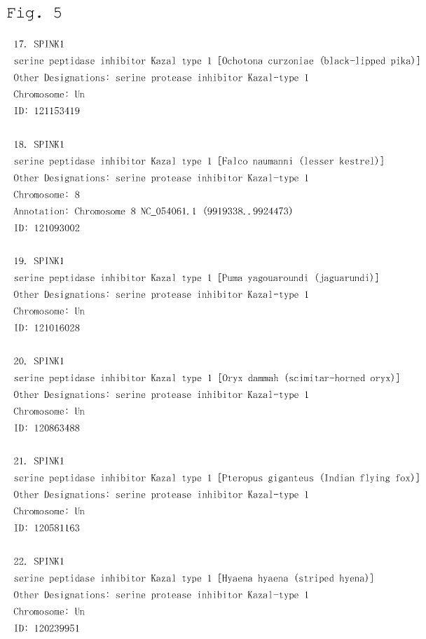

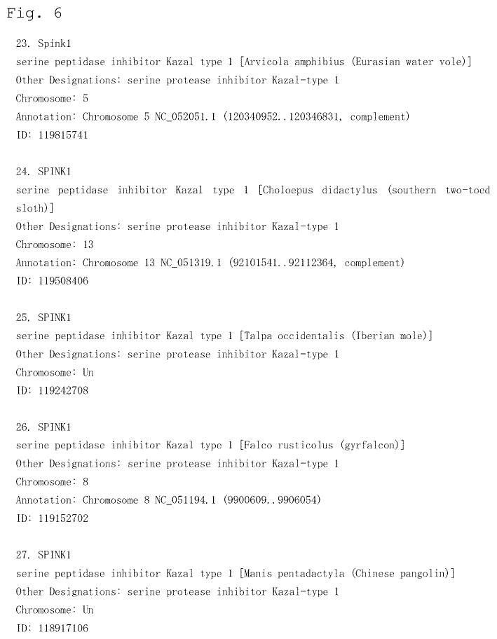

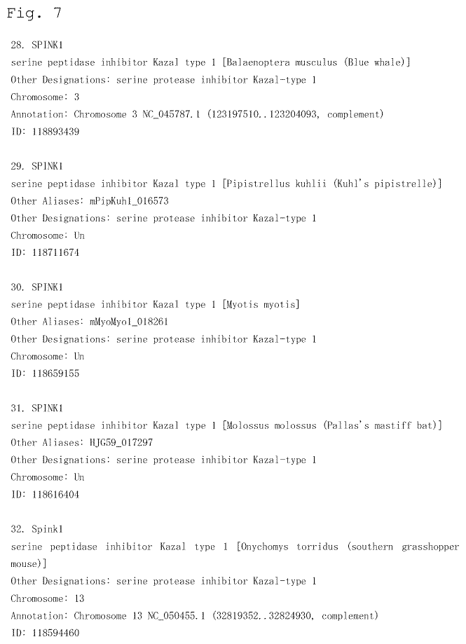

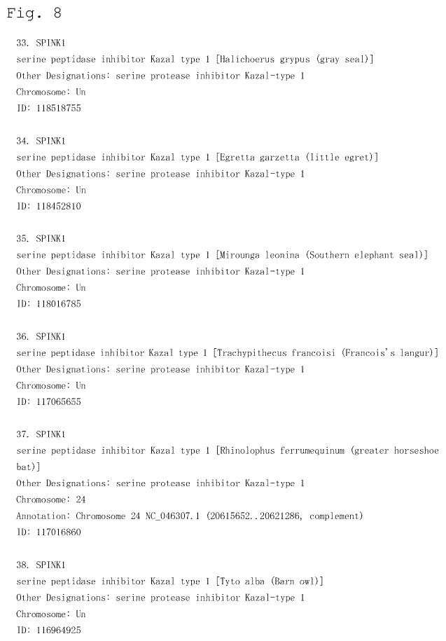

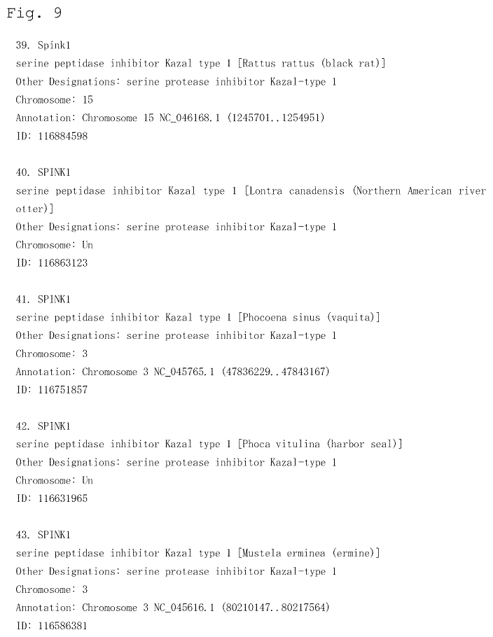

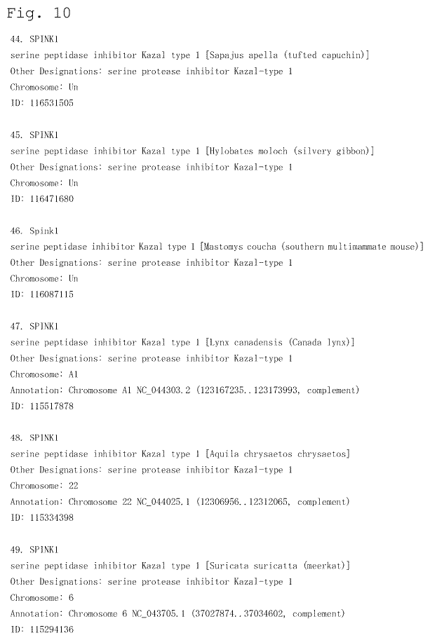

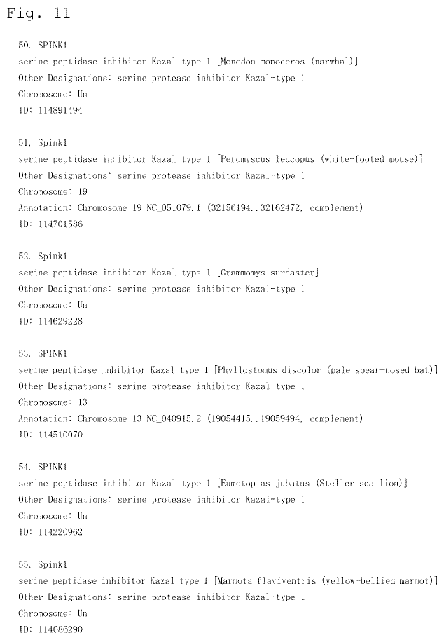

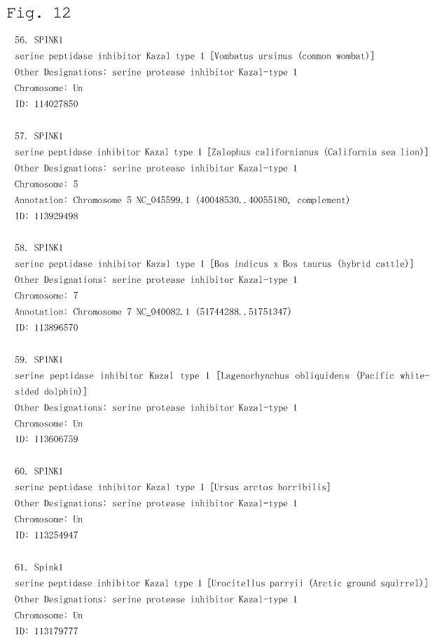

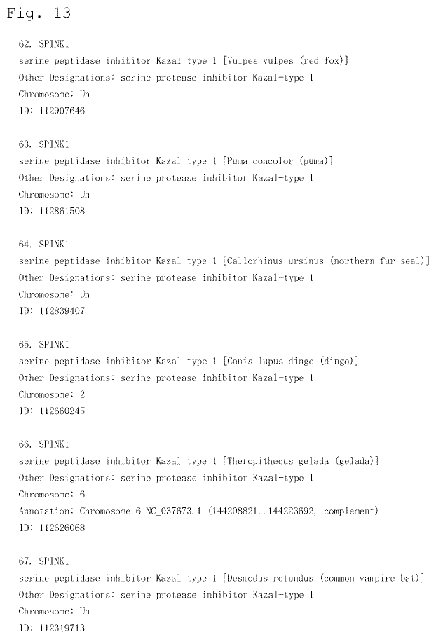

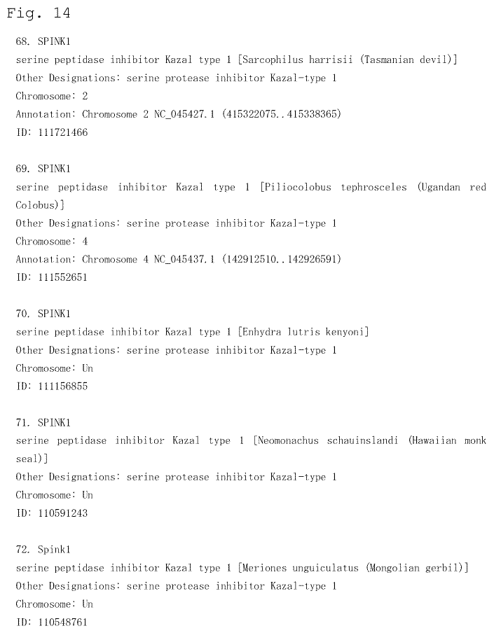

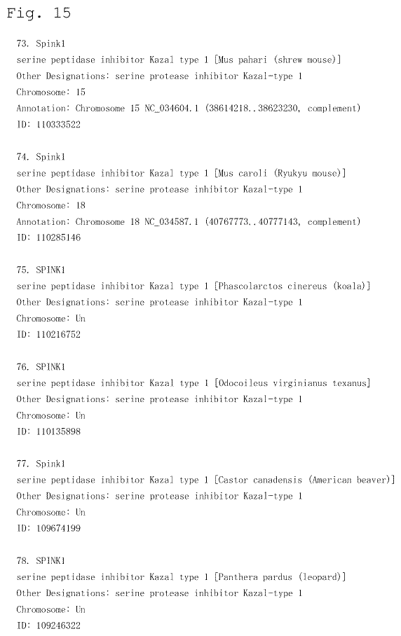

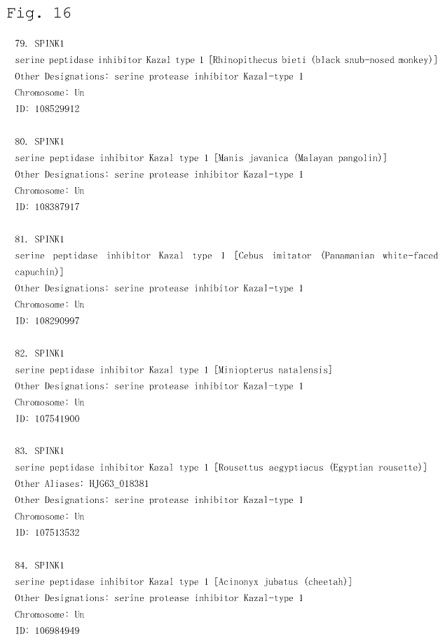

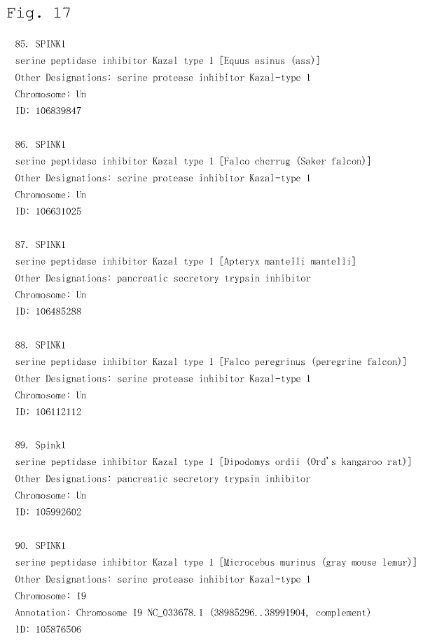

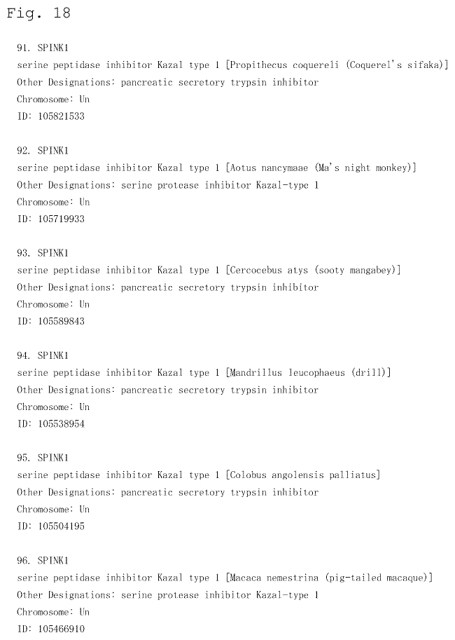

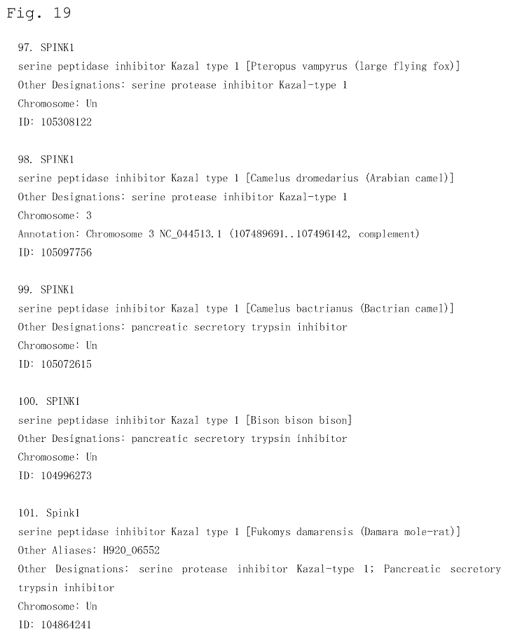

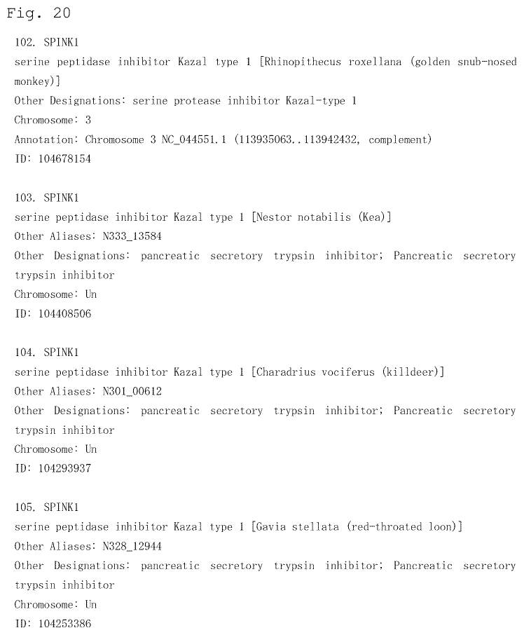

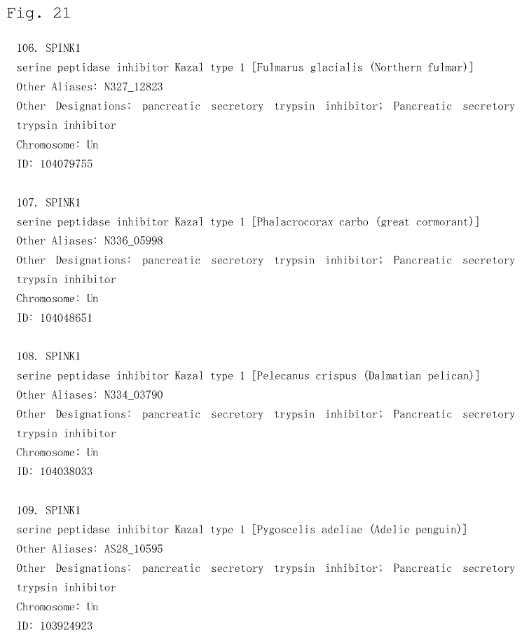

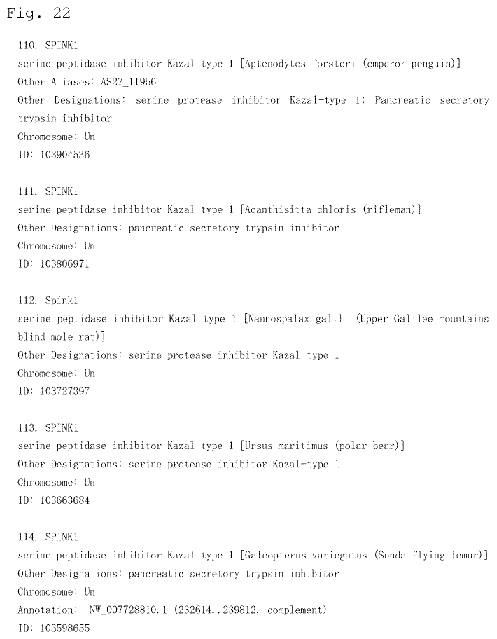

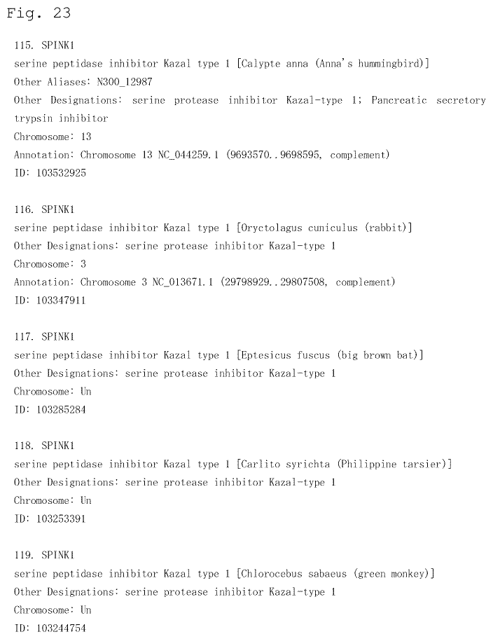

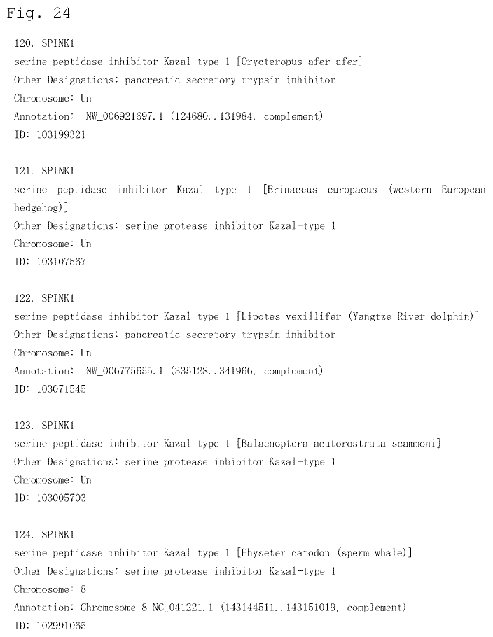

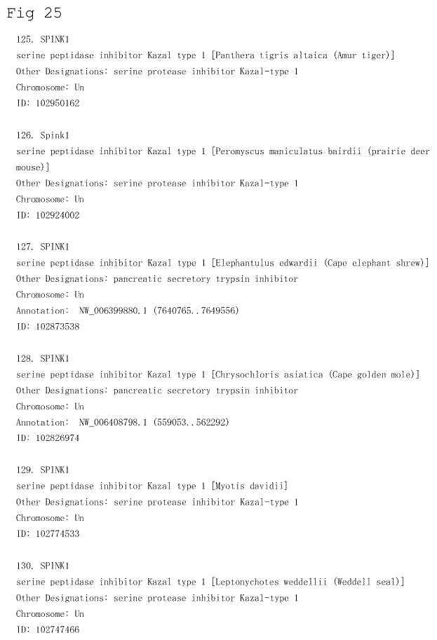

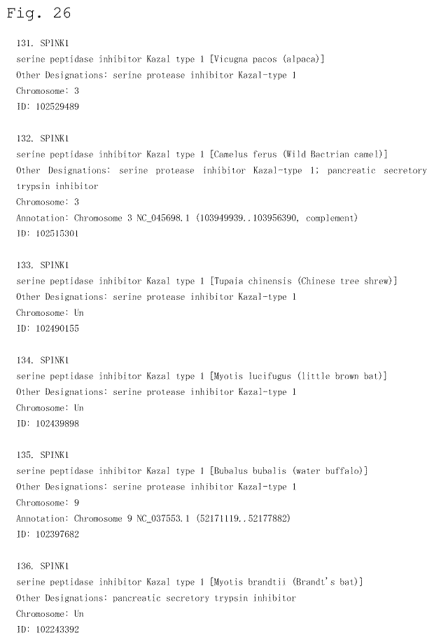

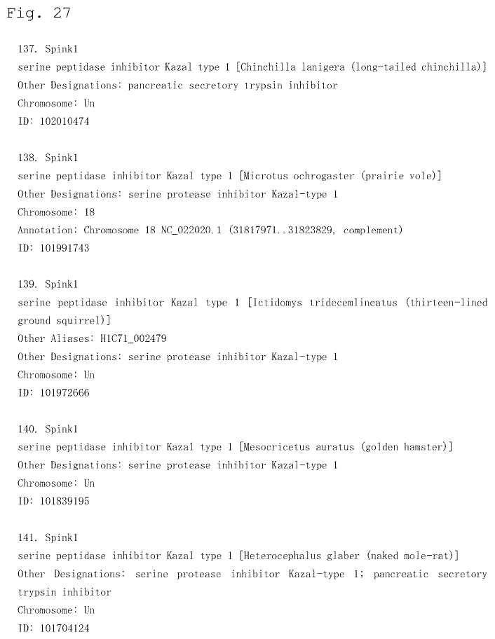

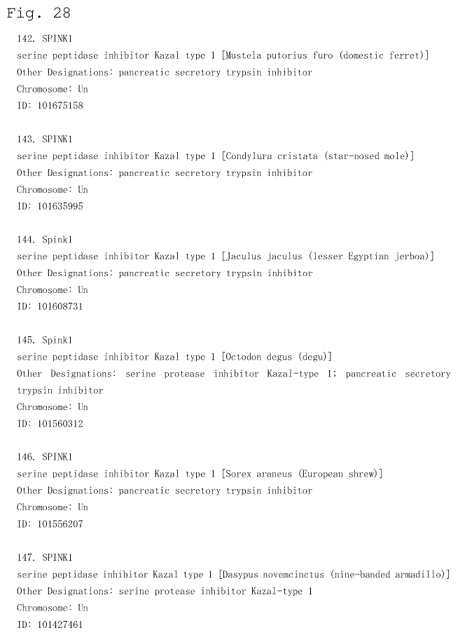

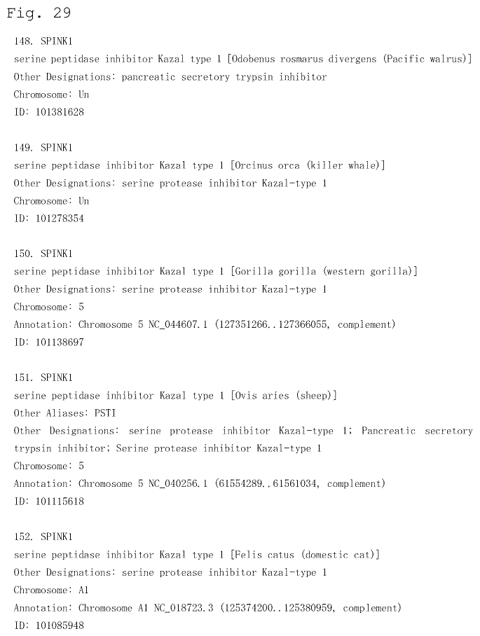

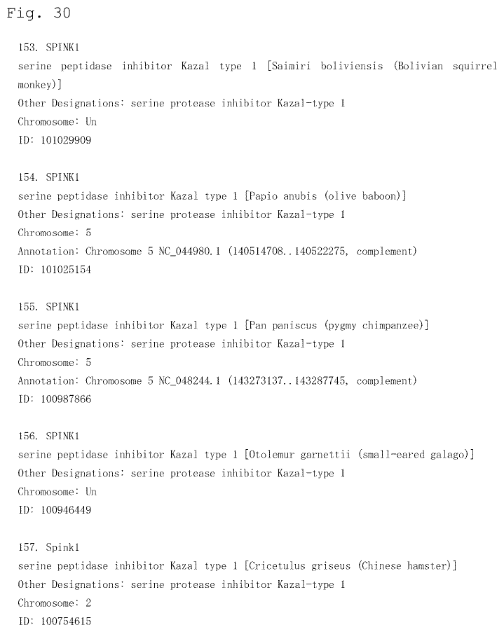

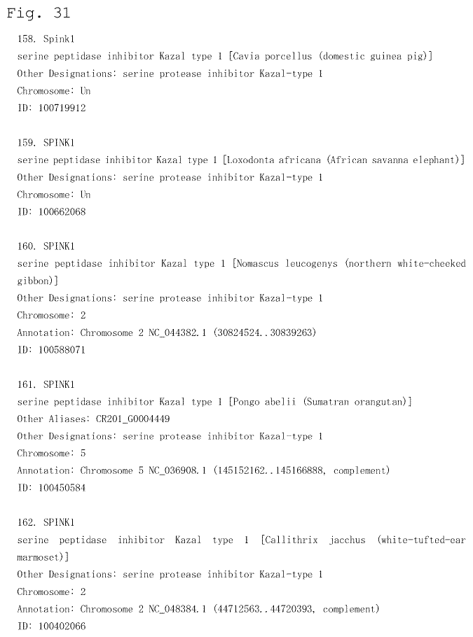

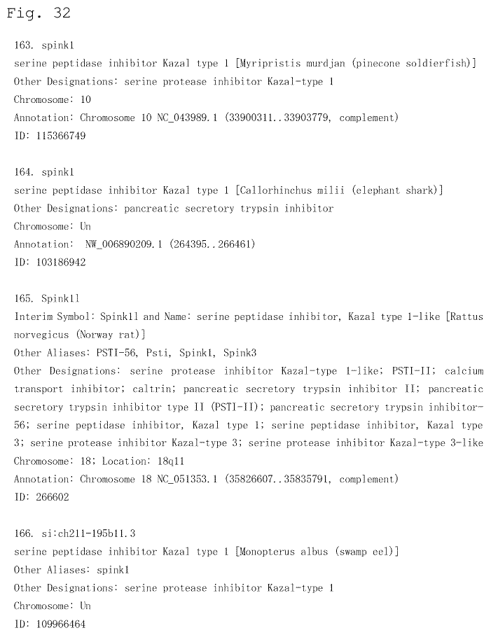

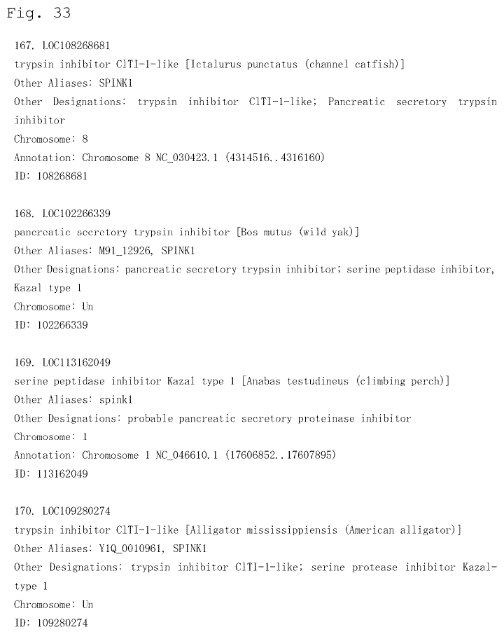

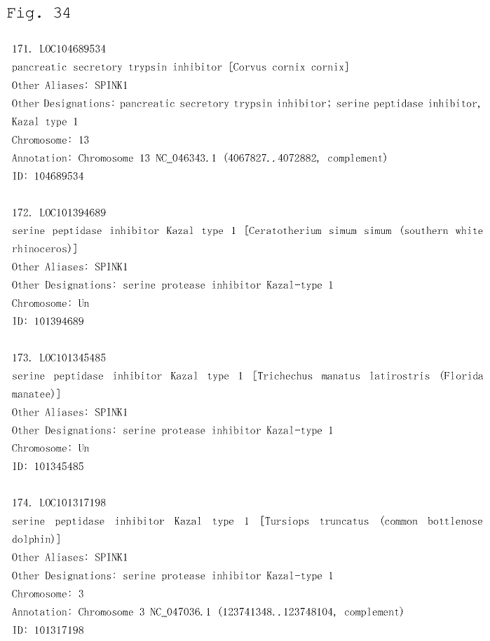

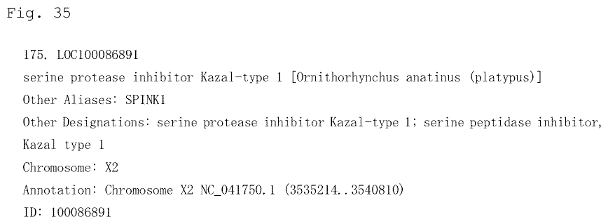

SPINK1 is known as a serine protease inhibitor Kazal type I. With no intention to limit the present disclosure, SPINK1 can be known, for example, from known databases, such as those shown in FIGS. 1 to 35. FIGS. 1 to 35 are examples of reports on SPINK1 of humans or other species shown in the NCBI. The report on SPINK1 (Homo sapiens (human) ID: 6690), which is described first among the reports shown in FIGS. 1 to 35, is information updated in May 2021. The others are examples of SPINK1 of rats or other species reported at the same point in time, i.e., in May 2021. In the present disclosure, SPINK1 can be any SPINK1 and is not limited to the specific SPINK1 shown in these figures.

The sample collected from a subject is not limited as long as it is a target of measurement of hypoxia. Examples of such samples include blood, plasma, serum, saliva, milk, urine, stool, tear, sweat, hair, body hair, spinal fluid, bone marrow fluid, joint fluid, sputum, nasal discharge, sebum, lymph fluid, tissues and cells of pathological sites (e.g., tissue specimens and cell specimens, such as tumor tissues, tumor cells, and myocardial cells); preferably biological samples such as blood, plasma, serum, urine, milk, saliva, tissue specimens, and cell specimens; and more preferably biological samples, such as blood, plasma, serum, saliva, milk, and urine from the viewpoint of being less invasive to subjects. The pathological site, such as tumor tissue, can be, for example, benign or malignant. Collecting a sample from a subject can be performed in accordance with known procedures. Samples can be used alone or in a combination of two or more.

Examples of subjects include mammals such as humans, monkeys, mice, rats, rabbits, goats, sheep, horses, guinea pigs, hamsters, dogs, cats, monkeys, and elephants; preferably humans, monkeys, mice, and rats; and more preferably humans.

As long as the amount of SPINK1 in a sample can be measured, the procedures for measuring the amount of SPINK1 in the sample are not limited. The measurement can be performed according to known procedures, and the amount of SPINK1 can be measured directly or indirectly. With no intention to limit the present disclosure, examples of SPINK1 measured include mRNA precursors, mRNA, proteins, and activity. As long as the amount of SPINK1 can be reflected, any object that changes in accordance with changes in the amount of SPINK1 may be used as the target of measurement, and the amount of SPINK1 may be indirectly measured based on, for example, the measurement results of the object.

With no intention to limit the present disclosure, as long as the amount of SPINK1 can be measured, the procedures for the measurement are not limited. Examples include known gene detection methods and immunological methods; for example, for the measurement of mRNA precursors and mRNAs, examples include in situ hybridization, RT (Reverse Transcription)-PCR (including quantitative RT-PCR), microarray, RNA-Seq, and Northern blotting; and for the measurement of proteins, examples include immunostaining, Western blotting, ELISA (enzyme-linked immuno-sorbent assay), flow cytometry, dot blotting, and mass spectrometry. When a substance, activity, or the like that can reflect the amount of SPINK1 is measured, the procedures for the measurement can be appropriately determined according to the target of measurement. Samples may be directly subjected to SPINK1 measurement as is, or may be pretreated as necessary.

With no intention to limit the present disclosure, examples of primers for quantitative RT-PCR include SPINK1 Human qPCR Primer Pair (NM_003122) (Catalog #24833) produced by OriGene Technologies, Inc. With no intention to limit the present disclosure, examples of antibodies include Anti-SPINK1/P12 antibody (catalog #ab183034) produced by Abcam PLC. Further, examples of ELISA kits include Human SPINK1 DuoSet ELISA (catalog #DY7496-05) produced by R & D Systems. Thus, primers, antibodies, and the like that are already known for the measurements described above can be appropriately used.

In the present disclosure, the measurement of the amount of SPINK1 further means detection. That is, the measurement of the amount of SPINK1 may be to comprehend whether SPINK1 is present or absent in a sample, or may be to comprehend the amount of SPINK1 in a sample. In the present disclosure, the amount measured may be an absolute amount or a relative amount.

The measurement method of the present disclosure may further comprise the step of setting a reference value for the amount of SPINK1 and comparing the reference value with the amount of SPINK1 (measured value) in a sample actually measured in the manner as described above.

The reference value may be arbitrarily set according to the purpose. For example, the reference value may be the amount of SPINK1 in a sample collected from a person whose oxygen concentration is a normal value (e.g., a sample derived from a healthy person, a sample derived from a person who has no pathological condition that causes a decrease in oxygen concentration). In this case, if the amount of SPINK1 (measured value) in a sample collected from a subject is higher than the reference value, the subject from whom the sample was collected is determined to have a site with an oxygen concentration lower than normal oxygen concentration; and if the measured value is as low as or lower than the reference value, the subject from whom the sample was collected is determined to be normal in terms of oxygen concentration.

Alternatively, the reference value may be the amount of SPINK1 in a sample collected from a person whose oxygen concentration is lower than a normal value (e.g., a sample derived from a person with a pathological condition). In this case, if the measured value is as high as or higher than the reference value, the subject from whom the sample was collected is determined to have a site with an oxygen concentration as low as or lower than that of the provider of the reference value (e.g., a person with a pathological condition), or have more sites with such an oxygen concentration than the provider of the reference value. If the measured value is lower than the reference value, the subject from whom the sample was collected can be determined to have no site with an oxygen concentration lower than that of the provider of the reference value or have fewer sites with an oxygen concentration as low as or lower than that of the provider of the reference value.

With no intention to limit the present disclosure, such a reference can be used to determine the degree of hypoxia in a subject. As long as this determination can be made, the measured value and the reference value are not limited. The measurement is preferably made by using the same type of sample (for example, if the sample used to obtain a measured value is blood, the sample used to obtain the reference value is also blood). The measurement of the amount of SPINK1 is also performed by using the same measurement procedure. The reference value may also be a value obtained by pre-measuring a sample collected from a subject.

In one embodiment of the measurement method of the present disclosure, if SPINK1 is detected in a sample, it can be determined that the subject from whom the sample was collected has a high probability of having a site in a hypoxic state (an area with a reduced oxygen partial pressure). If SPINK1 is not detected, it is determined that the subject from whom the sample was collected has no site in a hypoxic state.

In one embodiment of the measurement method of the present disclosure, when a sample has a larger amount of SPINK1, the subject from whom the sample was collected is determined to have a site with a lower concentration, or have more sites in a hypoxic state; and when a sample has a smaller amount of SPINK1, the subject from whom the sample was collected is determined to have an oxygen concentration closer to the normal oxygen concentration, or have fewer sites in a hypoxic state.

Therefore, the measurement method of the present disclosure may optionally further comprise such a determination step etc. Note that the relationship between the degree of hypoxia (oxygen concentration) and the severity of a pathological condition (exacerbation) may vary depending on the type of pathological condition (disease) and other factors. Therefore, the reference value may be appropriately determined depending on the type of pathological condition (disease) and other factors. The reference value may be an absolute value or a relative value.

According to the measurement method of the present disclosure, it is possible to know the presence or absence of SPINK1 and/or the amount of SPINK1 in a sample. Therefore, the measurement method is useful in that the presence or absence of SPINK1 and/or the amount of SPINK1 in a sample can be comprehended. As shown in the Examples below, the present inventors found that there is a correlation between hypoxia and the amount of SPINK1. The present inventors further found that the lower the oxygen concentration, the larger the amount of SPINK1 expressed, secreted, etc. Thus, it can be understood that the amount of SPINK1 in the living body tends to increase in a hypoxia concentration-dependent manner (the lower the oxygen concentration and/or the more hypoxia sites (areas) in the living body, the larger the increase in the amount of SPINK1). Further, it is said that the lower the oxygen concentration, the worse or the more refractory the pathological condition (the higher the risk of exacerbation). It can be said from this that the degree of hypoxia in a subject can be known by measuring the amount of SPINK1 in a sample collected from a subject. Accordingly, it can also be said that areas at a reduced oxygen partial pressure in a subject (e.g., the presence or absence of such hypoxic areas and the degree of hypoxia) can be measured (detected). From this point, the present disclosure may also be said to provide a method for measuring hypoxia in a subject based on the amount of SPINK1 in a sample.

Thus, the measurement method of the present disclosure is also useful in that one can know, for example, the degree of hypoxia in a subject, based on the amount of SPINK1 in a sample. Further, as described below, the method is also useful in that an exacerbated pathological condition or the risk of exacerbation can be predicted by using the amount of SPINK1 as an indicator. This prediction is also useful in investigating and performing a treatment for a pathological condition according to the subject. The pathological condition is explained as described below.

### Method for Predicting the Risk of Exacerbation of a Pathological Condition

The present disclosure further includes a method for predicting the risk of exacerbation of a pathological condition, comprising the step of measuring the amount of SPINK1 in a sample collected from a subject.

The subject, sample, SPINK1, measurement, and the like are all explained as described above in the measurement method. It is conventionally said that the lower the oxygen concentration, the worse the pathological condition. In the prediction method of the present invention, it can be predicted from this that the larger the amount of SPINK1 in a sample, the higher the risk of exacerbation of a pathological condition in the subject; and the smaller the amount of SPINK1 in a sample, the lower the risk of exacerbation of a pathological condition in the subject. With no intention to limit the present disclosure as stated above, preferable examples of the amount of SPINK1 to be measured in the prediction method include the mRNA level and the protein level, and more preferably the protein level.

Examples of pathological conditions include tumors and ischemic diseases such as ischemic heart diseases (e.g., angina pectoris and myocardial infarction) and ischemic brain diseases (e.g., cerebral infarction); and preferably tumors, myocardial infarction, and cerebral infarction. Examples of tumors include brain cancer, pharyngeal cancer, laryngeal cancer, esophageal cancer, lung cancer, stomach cancer, colon cancer, liver cancer, gallbladder cancer, kidney cancer, bladder cancer, prostate cancer, testicular cancer, breast cancer, uterine cancer, cervical cancer, ovarian cancer, bone tumor, and skin cancer. Such pathological conditions (pathological sites, not in a healthy condition) are usually considered to be in a hypoxic state. It can be said that the lower the oxygen concentration, the worse or the more refractory the pathological condition, that is, the higher the risk of exacerbation. Thus, in the present disclosure, the risk of exacerbation means the degree of progression or refractory level of a pathological condition; and further means malignancy (malignancy level), for example, in the case of tumors. It can be said that the higher the risk of exacerbation, the further the pathological condition is progressing, and the more refractory (harder to cure) the pathological condition. With no intention to limit the present disclosure, examples of the exacerbation of a pathological condition include radiation resistance of tumor tissue, which is refractory.

In the present disclosure, hypoxia is not limited as long as it is within the range of hypoxia known in this technical field. As can be understood from the Examples described below, it can be said that as the oxygen concentration decreases to 0.1% or lower, the amount of SPINK1 tends to increase. It can also be said that when the oxygen concentration is at a lower level, the amount of SPINK1 tends to significantly increase. In the present disclosure, hypoxia can be explained as an oxygen concentration being below the normal oxygen partial pressure in each organ, and the oxygen concentration is preferably, for example, in the range of 5% or less, 3% or less, 1% or less, or 0.1% or less. The lower limit of the oxygen concentration is theoretically 0%. The oxygen concentration is a value obtained by measurement procedures usually used in this technical field. This explanation is also applicable to the measurement method described above.

According to the prediction method, the risk of exacerbation of a pathological condition can be predicted based on the amount of SPINK1 measured.

Thus, in one embodiment of the prediction method of the present disclosure, it is determined that the larger the amount of SPINK1 in a sample, the higher the risk of exacerbation of a pathological condition in a subject from whom the sample was collected (there is a high probability that the pathological condition is progressing, is highly malignant, or is refractory) (that is, a high risk of exacerbation of a pathological condition is predicted); and it is determined that the smaller the amount of SPINK1, the lower the risk of exacerbation of a pathological condition in a subject from whom the sample was collected (that is, a low risk of exacerbation of a pathological condition is predicted).

The prediction method of the present disclosure may further comprise the step of setting a reference value and comparing the reference value with the amount of SPINK1 in a sample measured in the manner as described above (the measured value). The reference value may be arbitrarily set according to the purpose. For example, in a case in which the amount of SPINK1 in a sample that has been already determined to have a low risk of exacerbation is used as a reference value, if the amount of SPINK1 (the measured value) in a sample collected from a subject is higher than the reference value, then the subject from whom the sample was collected is determined to have a high probability of having exacerbation of a pathological condition; and if the measured value is as low as or lower than the reference value, then the subject from whom the sample was collected is determined to have a low probability of having exacerbation of a pathological condition. Further, for example, in the case in which the amount of SPINK1 in a sample that has been already determined to have a high risk of exacerbation is used as a reference value, if the amount of SPINK1 (the measured value) in a sample collected from a subject is as high as or higher than the reference value, then the subject from whom the sample was collected is determined to have a high probability of having exacerbation of a pathological condition; and if the measured value is smaller than the reference value, then the subject from whom the sample was collected is determined to have a low probability of having exacerbation of a pathological condition. In these prediction methods as well, the measured value and the reference value are not limited. However, as stated above, preferably, the measured value and the reference value are values obtained by using the same type of sample (for example, if the sample used to obtain the measured value is blood, then the sample used to obtain the reference value is also blood), and the amount of SPINK1 is measured in accordance with the same measurement procedures. The reference value may also be a value obtained by pre-measuring a sample collected from a subject.

In one embodiment of the prediction method according to the present disclosure, if SPINK1 is detected in a sample, it can be determined that a subject from whom the sample was collected has a high probability of having a site in a hypoxic state; and if SPINK1 is not detected in a sample, a subject from whom the sample was collected is determined to have no site in a hypoxic state. In the former case, a high risk of exacerbation of a pathological condition is predicted; in the latter case, a low risk of exacerbation or no pathological condition (pathological site) is predicted.

Thus, the prediction method of the present disclosure may optionally further comprise, for example, the step of making such a determination.

The relationship between the degree of hypoxia (oxygen concentration) and the severity of a pathological condition (exacerbation) may vary depending on the type of pathological condition (disease) and other factors. Therefore, the reference value may be appropriately determined depending on, for example, the condition of a subject from whom a sample is collected, the type of pathological condition (disease), and the severity of the pathological condition. Thus, the reference value may be appropriately determined by considering the amount of SPINK1 in a sample derived from, for example, a healthy person or a patient already determined to have a pathological condition. The reference value may be determined by a method different from that of the present disclosure or may be determined beforehand by the method of the present disclosure, or may be based on a value shown in databases regarding pathological conditions, oxygen concentration and/or the amount of SPINK1. The reference value may be a value derived from a subject who is subjected to prediction, or may be a value derived from a third party who is different from the subject. The reference value may also be an absolute value or a relative value. This explanation also applies to the measurement method described above.

Further, in the prediction method or the measurement method, a subject from whom a sample is collected may be a subject having some pathological condition (disease), may be a subject suspected of having a pathological condition (disease), may be a subject whose pathological condition (disease) is unknown, or may be a subject with no pathological condition (disease).

With no intention to limit the present disclosure, as stated above, it is said that the lower the oxygen concentration, the worse the pathological condition. For example, in tumors, it is known that the oxygen concentration is low at or around a tumor site. It is also known that the lower the oxygen concentration, the more resistant the tumor is to radiation therapy. Further, a low oxygen concentration is known to promote cancer invasion etc. Myocardial infarction is mainly caused by lowering of the oxygen concentration due to obstruction of blood flow etc. Thus, various pathological conditions are known to closely relate to the oxygen concentration at or around the pathological site. Further, it is also necessary to select an appropriate therapeutic method according to the degree of hypoxia, as is typically shown by the fact that radiation resistance is observed in tumors having a reduced oxygen concentration.

Thus, according to the prediction method of the present disclosure, it is possible to predict the risk of exacerbation of various pathological conditions based on the presence or absence of SPINK1 in a sample and/or the amount of SPINK1 in a sample. The prediction method of the present disclosure is useful in this point. The prediction of the risk of exacerbation of a pathological condition is useful in considering whether or not a treatment is necessary for the subject and what therapeutic method is to be selected.

Accordingly, SPINK1 is useful as a biomarker for measuring hypoxia or for predicting the risk of exacerbation of a pathological condition. Thus, SPINK1 can also be referred to as a hypoxia marker. SPINK1 is explained as described above. With no intention to limit the present disclosure, preferable examples of biomarkers include SPINK1 mRNA and SPINK1 protein. When this marker is used, blood or the like can also be used as measurement samples. Therefore, this marker can also be used, for example, as a blood marker.

Since the measurement method and the prediction method described above can monitor the amount of SPINK1 in a subject, these methods are also useful for determination of a therapeutic effect, prognosis prediction, etc., based on changes in the amount of SPINK1. The determination of a therapeutic effect includes a determination of whether or not the treatment is efficacious. For example, if an increase in the amount of SPINK1 in a sample is suppressed or the amount of SPINK1 decreases, the treatment is determined to be efficacious. If an increase in the amount of SPINK1 is not suppressed or the amount of SPINK1 increases, the treatment is determined to be inefficacious. The prognosis prediction is a determination of whether the prognosis is good or not. For example, when the amount of SPINK1 in the sample is low and if an increase in the amount of SPINK1 is suppressed or the amount of SPINK1 is reduced, the prognosis is predicted to be good. When the amount of SPINK1 is high and if an increase in the amount of SPINK1 is not suppressed or the amount of SPINK1 is increased, the prognosis is predicted to be bad. Thus, the determination of therapeutic effect and prognosis prediction can be made based on the measurement method and prediction described above.

### Composition for Measuring Hypoxia or Predicting the Risk of Exacerbation of a Pathological Condition

The present disclosure further includes a composition for measuring hypoxia or for predicting the risk of exacerbation of a pathological condition, the composition comprising a SPINK1 detection reagent.

The SPINK1, the measurement of hypoxia, and the prediction of the risk of exacerbation of a pathological condition are as described above.

The SPINK1 detection reagent is not limited as long as it can detect SPINK1. Examples include primers, probes, antibodies, aptamers (e.g., nucleic acid aptamers), compounds (e.g., natural compounds and synthetic compounds), and the like capable of specifically detecting SPINK1. Usable antibodies can be any antibody, such as polyclonal antibodies, monoclonal antibodies, and fragments thereof (e.g., Fab, Fab′, and F (ab′) 2). The detection reagent to be used may any reagent that can directly or indirectly detect the mRNA precursor, mRNA, protein, activity, etc., of SPINK1, as described above, and is not limited as long as the presence etc. of SPINK1 can be comprehended.

These reagents may also be labeled with, for example, an enzyme or a fluorescent dye, may bind to other proteins, etc., or may be immobilized on microplates, magnetic beads, or the like as needed.

The detection reagent may be a conventionally known SPINK1 detection reagent. With no intention to limit the present disclosure, examples include the primers, antibodies, and ELISA kits described above.

The composition may further contain optional components as long as such components do not interfere with the effect of the present disclosure. Examples of optional components include carriers, solvents, dispersants, emulsifiers, buffers, stabilizers, excipients, binders, disintegrants, lubricants, thickening agents, colorants, fragrances, chelating agents, pH adjusters, and preservatives. Such optional components may be used alone or in a combination of two or more. The amount of optional components added can be appropriately determined within the range that they do not interfere with the effects of the present disclosure.

The content of the SPINK1 detection reagent in the composition is not limited as long as SPINK1 can be detected. The content can be appropriately determined according to, for example, the type of primer, probe, antibody, or the like used. The composition may be in the form of a solid (e.g., powders, granules, and tablets), a semi-solid, or a liquid.

The detection using the composition can be performed with reference to known detection procedures according to, for example, the types of primers, probe, antibody, activity, target, etc. as described above. For example, the detection can be performed by the same procedures as in the measurement method described above.

The composition may be in the form of a kit. The kit may further contain, in addition to the composition, a buffer, a nucleic acid amplification reagent, reverse transcriptase, a substrate, a primary antibody, a secondary antibody, an instruction manual, an aptamer (e.g., nucleic acid aptamer), a compound (e.g., natural compounds and synthetic compounds), and the like usable for detection. For example, the instructions for use may describe the URL of a web page, reading code, or the like, or may be arranged such that the procedures for use, etc. can be obtained via a URL, reading code, etc. Thus, the present disclosure can also be said to provide a kit for measuring hypoxia or predicting the risk of exacerbation of a pathological condition.

The composition and the kit are useful in that the measurement and prediction described above can be easily performed.

### Screening Method

The present disclosure includes a method for screening a candidate substance for a therapeutic agent for a hypoxia-related pathological condition, the screening being performed using, as an indicator, whether or not the candidate substance can inhibit SPINK1.

As stated above, the present inventors found that SPINK1 can be used as an indicator in hypoxia-related pathological conditions, and further found that the larger the amount of SPINK1, the higher the risk of exacerbation of a pathological condition. Further, the present inventors found that inhibiting SPINK1 can mitigate the risk of exacerbation of a pathological condition (e.g., refractory degree), as shown in the Examples below. Thus, drugs capable of inhibiting SPINK1 are useful for improving hypoxia-related pathological conditions and suppressing the progression of hypoxia-related pathological conditions. Thus, screening for therapeutic agents for hypoxia-related pathological conditions can be performed based on whether or not a candidate substance can inhibit SPINK1.

Candidate substances are not particularly limited and can be any substance such as compounds (e.g., natural compounds, synthetic compounds, etc.), antibodies, and aptamers (nucleic acid aptamers). Candidate substances can be selected from known substances or can be newly synthesized, produced, etc.

The inhibition of SPINK1 includes, for example, being capable of reducing the expression level of SPINK1 mRNA precursor or SPINK1 mRNA, being capable of reducing the expression level of SPINK1 protein, being capable of reducing the secretion level of SPINK1 protein, and being capable of reducing the activity of SPINK1 protein. Further, the inhibition of SPINK1 can also be determined by using, as an indicator, for example, whether or not the activity of SPINK1 (the activity of inducing radiation resistance of cells, the activity of inducing antioxidant capacity of cells, the activity of inducing epidermal growth factor receptor (EGFR) signaling) can be inhibited. Thus, the inhibition of SPINK1 includes inhibition of expression and secretion, and inhibition of activity.

The hypoxia-related pathological condition is not limited as long as it is a condition in which hypoxia is a causative factor. Examples includes pathological conditions described above.

If a candidate substance can inhibit SPINK1, then the candidate substance is determined to have a high probability of being useful in treating a hypoxia-related pathological condition; and if a candidate substance cannot inhibit SPINK1, then the candidate substance is determined to have a high probability of not being useful in treating a hypoxia-related pathological condition. The screening method may further comprise this determination step.

Whether a candidate substance can inhibit SPINK1 or not can be appropriately determined with reference to known screening procedures according to the candidate substance.

In one embodiment of the screening, the screening method may comprise setting a control value for inhibition and comparing the inhibition achieved by using a candidate substance (a test value) with the control value. For example, when the SPINK1 inhibition level achieved by bringing a substance that does not have a SPINK1-inhibiting effect into contact with a sample is used as a control value, the SPINK1 inhibition level achieved by bringing a candidate substance into contact with a sample (a test value) is compared with the control value. If the test value is higher than the control value (i.e., if a large amount of SPINK1 is inhibited), the candidate substance is determined to have a high probability of inhibiting SPINK1. Based on this result, the candidate substance is determined to have a high probability of being useful in treating a hypoxia-related pathological condition. In contrast, if the test value is as low as or lower than the control value, the candidate substance is determined to have a low probability of inhibiting SPINK1. Based on this result, the candidate substance is determined to have a high probability of not being useful in treating a hypoxia-related pathological condition. Note that the SPINK1 inhibition level may be an absolute value or a relative value, and is not limited as long as the presence or absence of inhibition of SPINK1 and/or the degree of inhibition of SPINK1 can be known. The sample is not limited as long as the inhibition can be determined. For example, samples collected from the subjects described above may be used.

In one embodiment of the screening, the value achieved by using a substance that is already known to be useful in the treatment of a hypoxia-related pathological condition or a substance that is already known to have a SPINK1-inhibiting effect may be used as a control value. For example, when the SPINK1 inhibition level achieved by bringing such a substance into contact with a sample is used as a control value, the SPINK1 inhibition level achieved by bringing a candidate substance into contact with a sample (a test value) is compared with the control value. If the test value is as high as or higher than the control value, the candidate substance is determined to have a high probability of inhibiting SPINK1. Based on this result, the candidate substance is determined to have a high probability of being useful in treating hypoxia-related pathological conditions. If the test value is lower than the control value, the candidate substance is determined to have a low probability of inhibiting SPINK1. Based on this result, the candidate substance may be determined to have a high probability of not being useful in treating a hypoxia-related pathological condition. In this case as well, the inhibition level (inhibition amount) and the sample are explained as described above.

The screening method is useful, for example, in searching, creating, or determining a therapeutic agent for a hypoxia-related pathological condition.

### Composition for Ameliorating Hypoxia-Related Pathological Condition

The present disclosure further includes a composition for ameliorating a hypoxia-related pathological condition, the composition comprising a SPINK1 inhibitor.

As shown in the Examples below, the present inventors found that inhibiting SPINK1 can mitigate the refractory degree of a hypoxia-related condition. Thus, the present disclosure also provides a composition for ameliorating a hypoxia-related pathological condition, the composition comprising a SPINK1 inhibitor.

The SPINK1 inhibitor is not limited as long as it can inhibit SPINK1. Examples include compounds (e.g., natural compounds and synthetic compounds), antibodies, and aptamers (nucleic acid aptamers); preferably antibodies; and more preferably SPINK1 neutralizing antibodies. The inhibitor may be one capable of inhibiting SPINK1 as stated above. The inhibition of SPINK1 can be explained in the same manner as described above.

The content of the SPINK1 inhibitor in the composition is not limited and can be appropriately determined according to, for example, the type of compound, antibody, aptamer, or the like used and inhibition capability. The composition may be in the form of a solid (e.g., powders, granules, or tablets), a semi-solid, or a liquid. The route of administration is also not limited and may be oral or parenteral. Examples of parenteral administrations include subcutaneous, intramuscular, intravenous, sublingual, oral mucosal, transrectal, transvaginal, intranasal, inhalation, spray, and dermal administrations.

The composition may further optionally contain pharmacologically acceptable components as long as such components do not interfere with the effect of the present disclosure. Examples of such pharmacologically acceptable components include solvents, pH adjusters, excipients, stabilizers, binders, disintegrants, lubricants, colorants, fragrances, and preservatives. Such components may be used alone or in a combination of two or more. The amounts of components optionally incorporated may also be appropriately selected within the range that they do not interfere with the effect of the present invention.

The composition for amelioration is useful for ameliorating a hypoxia-related pathological condition. The hypoxia-related pathological condition can be explained as described above in the pathological condition.

### EXAMPLES

The present disclosure is described below in more detail by showing Examples. However, the present disclosure is not limited to the Examples.

### Test Example 1

### 1-1. Test Procedure

Human cervical cancer-derived cell line HeLa was sown into a cell culture dish (1×10⁵ per well of the 6-well cell culture plate) and cultured overnight in a CO₂ incubator at a normal oxygen concentration (20%). After the cells were then cultured in a CO₂ incubator set to an oxygen concentration shown in FIG. 36 for the periods of time shown in FIG. 36, the total RNA was collected using Sepasol RNA I Super G (produced by Nacalai Tesque, Inc.) A reverse transcription reaction and quantitative PCR experiments for SPINK1 and CA9 were performed. A quantitative PCR experiment using ACTB as an internal standard was also performed at the same time.

### 1-2. Results

FIG. 36 shows the results. FIG. 36 (a) shows the expression level of SPINK1 mRNA after 48 hours of exposure of HeLa cells to each oxygen concentration; FIG. 36 (b) shows the expression level of CA9 mRNA after 48 hours of exposure of HeLa cells to each oxygen concentration; and FIG. 36 (c) shows the expression level of SPINK1 mRNA after 12, 24, and 48 hours of exposure of Hela cells to an oxygen concentration of 20% or 0.1%.

As shown in FIG. 36 (a) and FIG. 36 (b), the expression level of SPINK1 mRNA and the expression level of CA9 mRNA significantly increased under exposure to an oxygen concentration of 0.1%. In particular, the expression level of CA9, which is conventionally known as a hypoxia marker, tended to increase gradually as the oxygen concentration decreased to 3%, 1% and 0.1%, whereas the expression level of SPINK1 at an oxygen concentration of 3% or 1% was as low as it was at an oxygen concentration of 20%, with a sharp increase in the expression level of SPINK1 at an oxygen concentration of 0.1%. Further, as shown in FIG. 1 (c), it was found that as the time of exposure to a low oxygen concentration environment increased to 12 hours, 24 hours, and 48 hours, the expression level of SPINK1 mRNA increased. This confirmed that the SPINK1 level increases in a hypoxia stimulus-dependent manner.

### Test Example 2

### 2-1. Test Procedure

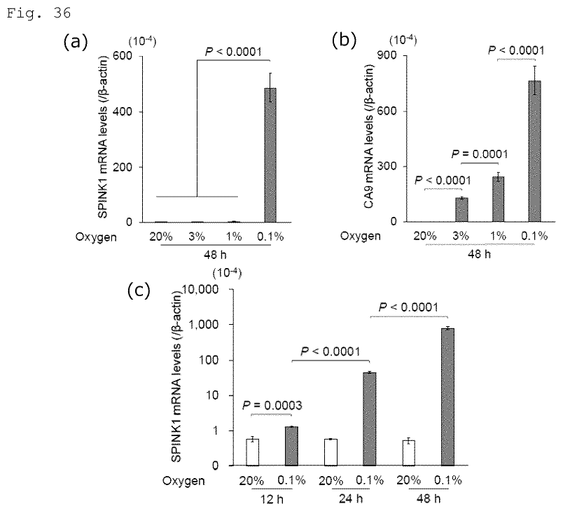

Human cervical cancer-derived cell line HeLa, human prostate cancer-derived cell line DU 145, and human osteosarcoma-derived cell line U2OS were sown into cell culture dishes (1×10⁵ cells per well of a 6-well cell culture plate for quantitative RT-PCR and 1.5 to 2.0×10⁵ cells per well of a 6-well cell culture plate for ELISA assay), and cultured overnight in a CO₂ incubator at a normal oxygen concentration. Thereafter, after the cell lines were cultured for 48 hours in a CO₂ incubator set to the oxygen concentrations shown in FIG. 37, the cell culture fluid was collected and ELISA assay for SPINK1 was performed. At the same time, the total RNA was collected using Sepasol RNA I Super G (produced by Nacalai Tesque, Inc.), a reverse transcription reaction and a quantitative PCR experiment for SPINK1 were performed. A quantitative PCR experiment using ACTB as an internal standard was also performed at the same time.

### 2-2. Results

FIG. 37 shows the results. FIG. 37 shows the amount of SPINK1 mRNA in the cells (top row) and the amount of SPINK1 protein in the cell culture fluid (bottom row). As shown in FIG. 37, the SPINK1 mRNA expression level and the SPINK1 protein secretion level significantly increased at an oxygen concentration of 0.1% as compared with those at an oxygen concentration of 20%. These results confirm that the SPINK1 expression level and the SPINK1 secretion level increase in a hypoxia stimulus-dependent manner. Further, a correlation was observed between the SPINK1 mRNA level and the SPINK1 protein secretion level. Further, protein secretion in a hypoxic environment was also confirmed from Test Example 2.

### Test Example 3

### 3-1. Test Procedure

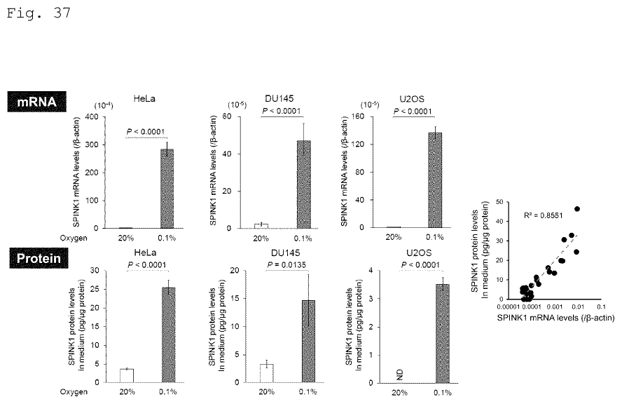

Tumor-bearing mice were prepared by subcutaneously transplanting a human cervical cancer-derived cell line HeLa into the right lower-leg thigh. Using the tumor-bearing mice as subjects, the blood vessel in the right lower-leg thigh was ligated to reduce the blood flow to the HeLa-transplanted tumor. After the lapse of time shown in FIG. 38, the transplanted tumor was removed. The total RNA was collected from the removed transplanted tumor by using Sepasol RNA I Super G (produced by Nacalai Tesque, Inc.). A reverse transcription reaction and a quantitative PCR experiment for SPINK1 were performed. A quantitative PCR experiment using ACTB as an internal standard was also performed at the same time. The total protein was also extracted from the removed transplanted tumor, and ELISA assay for SPINK1 was performed at the same time.

### 3-2. Results

FIG. 38 shows the results. As can be understood from FIG. 38, induction of the expression of SPINK1 mRNA and SPINK1 protein was observed in the biogenic tumor tissue exposed to a hypoxic environment by ligation. As the time of exposure to the hypoxic environment increased, both of the SPINK1 mRNA level and the SPINK1 protein level increased. This confirmed that the amount of SPINK1 increased in a hypoxia exposure-dependent manner.

Further, although not shown in the results, after transplantation of the tumor tissue (HeLa cells), blood vessels were ligated to reduce blood flow to the tumor, and tumor tissue was then collected. Quantitative reverse transcription PCT (RT-qPCR) was performed to measure and compare the expression level of SPINK1 mRNA in the cells and the expression level of known hypoxia marker CA9 mRNA. As a result, a high correlation (R2=0.95) was observed between their expression levels. From these results as well, SPINK1 could be understood to be useful as a hypoxia marker.

### Test Example 4

### 4-1. Test Procedure

Pimonidazole was administered to tumor-bearing mice that had been prepared by transplanting human cervical cancer-derived cell line HeLa. Sixty minutes after the administration, the transplanted tumor was removed. This tumor was fixed in a 10% neutral buffered formalin solution and then paraffin-embedded to prepare tumor sections. The paraffin sections were used in an immunohistochemical staining experiment using an anti-pimonidazole antibody (mouse monoclonal antibody; Hypoxyprobe, Inc., Catalog #HP2-1000) and anti-SPINK1 antibody (rabbit monoclonal antibody; EPR12696 (2), Abcam, Catalog #ab183034). After staining with each antibody, nuclei were stained using Hoechst 33342.

### 4-2. Results

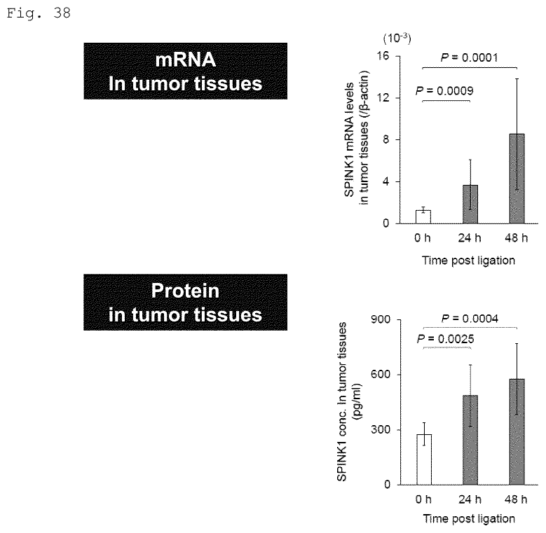

FIG. 39 shows the results. In FIG. 39, stained portions in the upper photo “Pimonidazole” indicate hypoxic areas; stained portions in the photo “SPINK1” indicate that SPINK1 protein is present; and stained portions in the photo “Hoechst” indicate that tumor cells are present. The lower photo in FIG. 39 is a superimposition of these photos. As can be seen from FIG. 39, SPINK1 protein was confirmed to be secreted from cells in a hypoxic state and be present in and around the cells.

Although not shown in the results, it was confirmed that even when anemia stimulation (hypoxia stimulation) is given to tumor-bearing mice prepared by transplanting human cervical cancer-derived tumor cell line HeLa, not only the SPINK1 mRNA expression in tumor cells increases but also the SPINK1 protein in blood plasma increases. These results confirmed that SPINK1 is useful, for example, as a blood marker.

### Test Example 5

### 5-1. Test Procedure

A SPINK1 Myc-tagged fusion protein expression vector or its empty vector was introduced into human cervical cancer-derived cell line HeLa and human prostate cancer-derived cell line DU145. After culturing under normal oxygen conditions for 48 hours, the cell culture fluid and the cell extract were collected and Western blotting was performed using anti-Myc tag antibody and anti-ß-Actin antibody. Forty-eight hours after the gene introduction, 0, 2, 4, or 6 Gy gamma ray irradiation was performed under normal oxygen conditions. The cells were then cultured for 14 days to perform a colony formation test.

### 5-2. Results

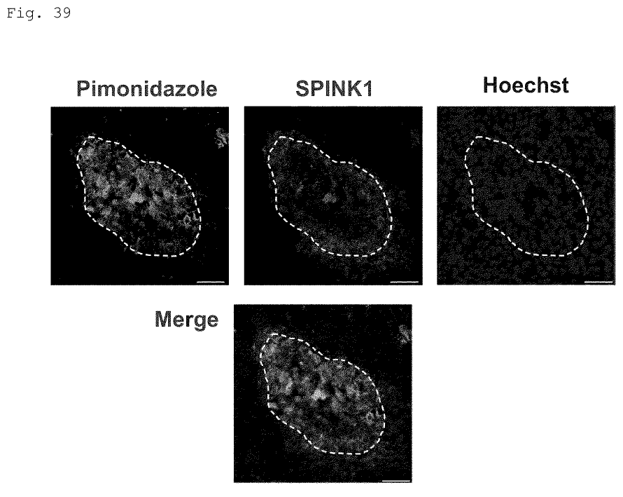

FIG. 40 shows the results. As can be understood from FIG. 40, it was confirmed that when a SPINK1 expression vector is introduced into Hela cells and DU145 cells, SPINK1 can be overexpressed in both of the Hela cells and DU145 cells. It was also confirmed that in both of Hela cells and DU145 cells, the overexpression of SPINK1 protein increases radiation resistance of the cells. Based on these results, it could be understood that SPINK1 contributes to exacerbation of a pathological condition and that SPINK1 can be an indicator of exacerbation of a hypoxia-related pathological condition.

### Test Example 6

### 6-1. Test Procedure

Human prostate cancer-derived cell line DU145 was cultured in serum-free medium under normal oxygen (20%) conditions for 24 hours. The medium was then replaced with normal medium (containing 5% serum) containing a recombinant SPINK1 protein at a final concentration of 100 ng/ml or with normal medium not containing the recombinant SPINK1 protein. The cells were cultured under normal oxygen conditions for 24 hours. The cells were then irradiated with 0, 2, 4, or 6 Gy gamma rays and cultured for 14 days. A colony formation test was then performed.

### 6-2. Results

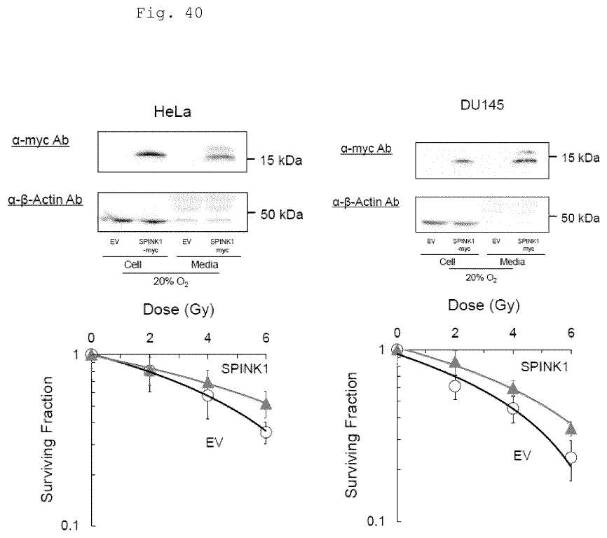

FIG. 41 shows the results. As can be understood from FIG. 41, it was confirmed that when the cells are irradiated in the presence of recombinant SPINK1 protein (rSPINK1 in FIG. 41), radiation resistance of the cells is induced. From these results as well, it could be understood that SPINK1 contributes to exacerbation of a pathological condition and that SPINK1 can be used as an indicator of exacerbation of a hypoxia-related pathological condition.

### Test Example 7

### 7-1. Test Procedure

SPINK1 expression vector (pCDH/SPINK1) and its empty vector (pCDH-EF1-MCS-IRES-Puro) were transfected into HEK293TN cells to prepare SPINK1-expressing lentivirus and empty lentivirus for a negative control. These viruses were infected with human prostate cancer-derived cell line DU145 and cultured in the presence of puromycin to create SPINK1 stable expressing cells (DU145/SPINK1) and negative control cells (DU145/EV).

These cells were transplanted subcutaneously into the right lower-leg thigh of immunocompromised mice (BALB/c nu/nu) to prepare tumor-bearing mice. The transplanted tumor was locally irradiated with 0 or 10 Gy gamma rays, and subsequent volume changes in the transplanted tumor were measured. The ratio of the tumor volume on each measurement date to the tumor volume before the irradiation was determined and the mean ± standard deviation was plotted (n=9 to 10 per group).

### 7-2. Results

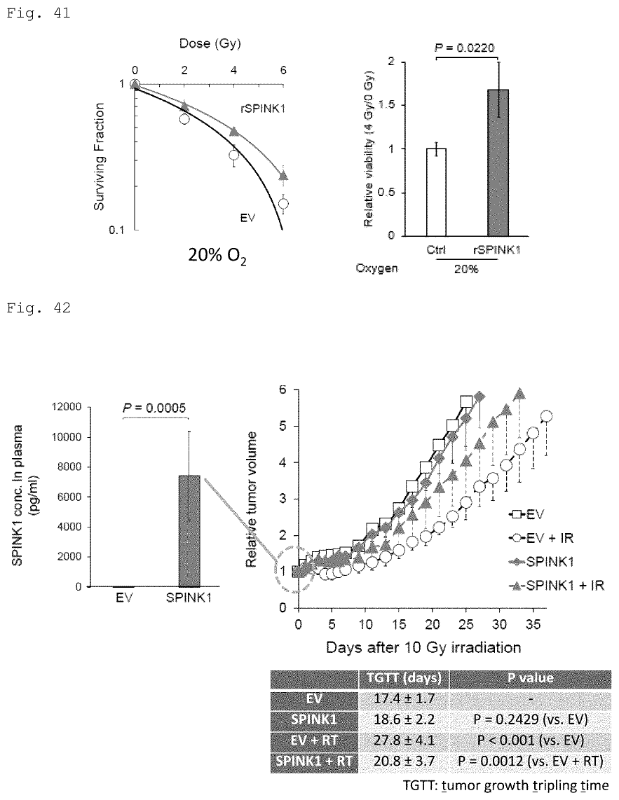

FIG. 42 shows the results. As can be understood from FIG. 42, it was confirmed that in vivo as well, the radiation resistance increases in the presence of SPINK1 protein. Based on these results, it could be understood that SPINK1 contributes to the worsening of a pathological condition in a hypoxic environment.

### Test Example 8

### 8-1. Test Procedure

Human prostate cancer-derived cell line DU145 was seeded into 96-well cell culture plates (4×10² cells per well in a 96-well plate when the dose of the subsequent irradiation is 0 Gy; and 1.2×10³ cells per well when the dose is 4 Gy) and cultured in a CO₂ incubator under a normal oxygen concentration overnight. Thereafter, the cells were cultured in serum-free medium for another 24 hours and then cultured in the presence or absence of recombinant SPINK1 purified protein and in the presence or absence of anti-SPINK1 antibody under normal oxygen conditions for 24 hours. The cells were then irradiated with 0 Gy or 4 Gy gamma rays and cultured under normal oxygen conditions for an additional 3 days. Viable cells were then quantified with Cell Count Reagent SF (produced by Nacalai Tesque, Inc., Catalog #07553-44).

### 8-2. Results

FIG. 43 shows the results. As can be understood from FIG. 43, radiation resistance of the cells was induced in the presence of recombinant SPINK1 purified protein. When SPINK1 neutralizing antibody was allowed to act on the protein, this effect disappeared. These results confirmed that inhibiting SPINK1 can mitigate radiation resistance of cancer cells. This confirmed that inhibiting SPINK1 can inhibit exacerbation of a pathological condition caused by a hypoxic area.

### Test Example 9

### 9-1. Test Procedure

Among the patients diagnosed with pancreatic cancer at Kyoto University Hospital during the period from January 2016 to December 2019, 21 surgical patients whose preoperative plasma was available were used as subjects. The concentration of SPINK1 was measured by ELISA using the preoperative plasma of each patient as samples, and the correlation between the overall survival rate and the SPINK1 concentration was evaluated. This study was conducted after full explanation and obtaining informed consent in accordance with ethical guidelines in order to confirm the will of the patients etc.

### 9-2. Results

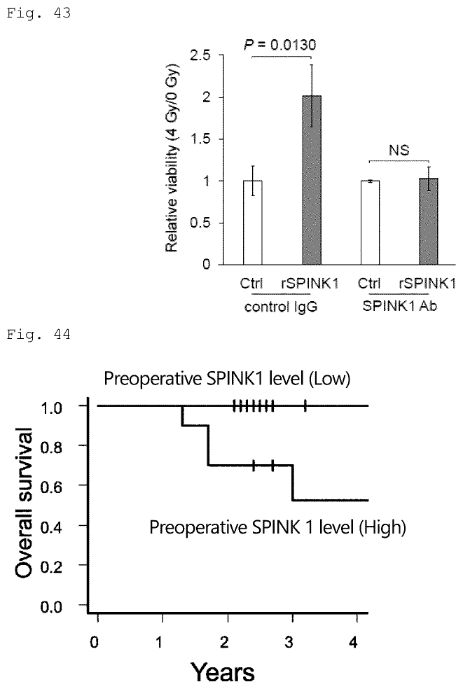

FIG. 44 shows the results. In FIG. 44, the overall survival rate is plotted on the ordinate, and the observation period (unit: year) is plotted on the abscissa, with the date on which the patient underwent tumor resection surgery being defined as 0. The median value of the SPINK1 concentration of the preoperative plasma was 27.6 μg/L (range: 20.5 to 185.5 μg/L). The median value of the observation period was 2.6 years (range: 1.3 to 5.0 years). Using the SPINK1 concentration of 27.6 μg/L as the threshold, the obtained preoperative SPINK1 concentration data were divided into two groups, i.e., a high-value group (High in FIG. 44) and a low-value group (Low in FIG. 44) to make a comparison therebetween in terms of the total survival rate. The results show that the 3-year survival rate of the high-value group was 52.5%(95% confidence interval: 15.0 to 80.4%) and the 3-year survival rate of the low-value group was 100%(NA-NA). Thus, the following tendency was observed: the higher the preoperative SPINK1 concentration, the lower the overall survival rate (p=0.0365). This confirmed that when SPINK1 is used as an indicator, the risk of exacerbation of a pathological condition can be predicted and the prognosis can also be predicted. These results confirmed that SPINK1 is thus useful as a biomarker for 10 hypoxia in a subject and the risk of exacerbation of a pathological condition.

1. A method for measuring hypoxia in a subject, comprising measuring the amount of SPINK1 (serine protease inhibitor Kazal type I) in a sample collected from the subject.

2. A method for predicting the risk of exacerbation of a pathological condition, comprising measuring the amount of SPINK1 in a sample collected from a subject.

3. The method according to claim 1, wherein the sample is at least one member selected from the group consisting of blood, plasma, serum, urine, milk, saliva, cell specimens, and tissue specimens.

4. The method according to claim 2, wherein the pathological condition is at least one member selected from the group consisting of tumors and ischemic diseases.

5. Use of SPINK1 as a biomarker for measuring hypoxia or predicting the risk of exacerbation of a pathological condition.

6. A composition for measuring hypoxia or predicting the risk of exacerbation of a pathological condition, the composition comprising a SPINK1 detection reagent.

7. A method for screening a candidate substance for a therapeutic agent for a hypoxia-related pathological condition, the screening being performed by using, as an indicator, whether or not the candidate substance can inhibit SPINK1.

8. A composition for ameliorating a hypoxia-related pathological condition, the composition comprising a SPINK1 inhibitor.
